(19) United States
(12) Patent Application Publication
(10) Pub. No.: US 2005/0070809 A1
(43) Pub. Date: Mar. 31, 2005

(54) System for regulating exercise and exercise network

(76) Inventor: John F. Acres, Corvallis, OR (US)

Correspondence Address: MARGER JOHNSON & MCCOLLOM, P.C., 1030 SW MORRISON STREET, PORTLAND, OR, 97205, US

(21) Appl. No.: 10/952,037

(22) Filed: Sep. 27, 2004

Related U.S. Application Data
(60) Provisional application No. 60/507,150, filed on Sep. 29, 2003.

(51) Int. Cl. A61B 5/02

(52) U.S. Cl. 600/508

(57) ABSTRACT

Disclosed is an automatic system for configuration, direction and recording of exercise program for an individual, based upon the attained heart rate of that person during an exercise period. Configuration data can be stored on an account accessible over a network. Configuration data can also be stored in an electronic device. The electronic device can provide information feedback to the user indicating the user's progress in a workout. In some embodiments the device can also send configuration commands to compatible exercise equipment according to heart rate or other exercise parameter.

### CROSS REFERENCE TO RELATED APPLICATIONS

This application claims priority from U.S. Provisional application 60/507,150, filed Sep. 29, 2003, which is incorporated herein by reference in its entirety.

### TECHNICAL FIELD

This disclosure relates to exercise, and, more particularly to automating the process of defining appropriate exercise levels based upon various factors of the person exercising.

### BACKGROUND OF THE INVENTION

Throughout this document, the term “her”, is used for illustrative purposes only and is meant to represent persons of both the male and female genders.

Exercise is an essential component of human health. Society has expended significant resources to determine optimal exercise parameters, particularly regarding duration and intensity of exercise. Exercise programs are difficult to configure and tracking an individual's performance manually is often confusing and time consuming. Such difficulties cause large segments of our population to exercise inefficiently or even dangerously.

A key metric of exercise is heart rate. Each individual has a maximum recommended heart rate that represents the approximate maximal work that person's heart can perform. This maximum heart rate can be measured through physical testing or can be calculated by methods well known to those skilled in the art of fitness medicine. The maximum recommended heart rate of each person varies according to several factors, such as genetics, gender, fitness level, general health and age. It is useful to recalculate or re-measure maximum heart rate on a regular basis. Once a maximum recommended heart rate is determined, fitness goals are set that specify a particular target duration of exertion at a heart rate that is some fraction of the maximum rate. For example, a general fitness program could be defined as three forty minute exercise sessions per week at an exertion levels between 70% and 75% of maximum heart rate and two exercise periods of thirty minutes per week at exertion levels of between 80% and 90% of maximum. To prepare the body for exercise and allow it afterward to return to normal levels, a fifteen-minute warm-up and fifteen-minute cool-down period may be recommended to precede and follow the exercise period. During the warm-up period, it is recommended that exercisers steadily increase exertion level to bring the heart rate to the desired level for the main exercise session. During the cool-down period, exertion level is slowly decreased until it reaches an appropriately low level for exercise cessation.

A range of devices is marketed today that monitor heart rate and display it as Beats Per Minute (BPM). One such product is the model S720 that is marketed by the Polar Electro Company of Finland. The S720 offers help for the exerciser in determining her maximum heart rate through use of a heart rate formula. The most common such formula is Maximum Heart Rate=220−Age. For a fifty-year-old person, this yields 220−50 or 170 BPM. In recent years, alternative formulae have emerged claiming to more accurately estimate maximum heart rate. One such formula is Maximum Heart Rate=205−(Age/2) with five counts added if the exerciser is male and five counts added if the exerciser is in relatively fit shape.

The S720 consists of a chest strap and wristwatch style monitor. The chest strap senses the electrical signals that are created with each heartbeat and wirelessly transmits a 5 KHz pulse for each heartbeat. The wristwatch monitor detects each heartbeat signal through inductive coupling, then measures and averages the time between each heartbeat, displaying the result on the wristwatch display in BPM.

In such a system, the exerciser occasionally glances at the wristwatch display to learn her current heart rate, and adjusts her exertion level to stay within a target exertion zone. Because it is sometimes inconvenient to look at a wristwatch display, the S720 can be configured to emit an audible chirping sound whenever the current heart rate falls outside the desired target zone. While the audible signals can be helpful, it is difficult to generate a significant volume of sound in a wristwatch-sized device. Polar and other vendors generally utilize a small piezo emitter that generates a high-pitched signal of typically between 2 and 4 KHz. Such tones, particularly at the volume that is readily generated by wristwatch-sized devices, are difficult to hear in high noise environments such as jogging on a roadside or exercising in a room with loud music, or by people with diminished hearing capacity. Also, the S720 emits identical tones whether the current heart rate is above or below the target heart rate zone. Therefore, the person exercising must visually examine information on the S270 wristwatch display to learn if she is at too high or too low a level of exertion.

The Polar S720 offers an infrared uplink to an IBM compatible personal computer (PC). Exercise data can be transferred from the wristwatch monitor to the PC for permanent storage and analysis. Such a system requires use of a PC along with an infrared interface and special application software. The exerciser must intervene with the process to affect the transfer of data by configuring the wristwatch to transmit, configuring the PC to receive, and selecting the data for transfer.

The S720 is also capable of transferring configuration commands from the PC through audio tones. Again, the PC and wristwatch monitor must each be configured for the transfer, and the specific data to be transferred must be specifically selected. Many exercisers find the process too complex for everyday use.

Embodiments of the invention address these and other limitations in the prior art.

### SUMMARY OF THE INVENTION

Embodiments of the invention eliminate the need for exercisers to calculate appropriate heart rate targets or to monitor heart rate exertion levels during exercise sessions. The current exertion level is compared against a previously calculated exertion goal and the user is instructed to increase, decrease, or maintain her exertion level. Embodiments of the invention also remove the need for record keeping by entirely automating the exercise monitoring process. Further, embodiments of the invention can automatically configure compatible exercise equipment, and automatically increase or decrease its exertion settings to bring the exerciser's heart rate to within the range of the previously established exertion goal, thereby removing that burden from the exerciser.

### BRIEF DESCRIPTION OF THE DRAWINGS

FIG. 1 is a block diagram illustrating an exercise network according to embodiments of the invention.

FIG. 2A is a functional block diagram illustrating an example embodiment of a bridge included in FIG. 1.

FIG. 2B is a functional block diagram illustrating another example embodiment of a bridge included in FIG. 1.

FIGS. 3-7 are functional block diagrams illustrating example embodiments of an electronic device illustrated in FIG. 1.

FIGS. 8 and 9 are a functional block diagrams illustrating exercise networks according to other embodiments of the invention.

FIG. 10 is a functional block diagram illustrating additional sensors that can be used in embodiments of the invention.

FIG. 11 is a functional block diagram illustrating another embodiment of a portion of an example exercise network.

FIGS. 12-15 are functional block diagrams illustrating further example exercise networks according to embodiments of the invention.

### DETAILED DESCRIPTION OF THE PREFERRED EMBODIMENTS

Embodiments of the invention provide a simple, enjoyable, and efficient system of exercise guidance that removes the need for technical knowledge and record keeping. Exercise intensity and duration are based upon the exerciser's current heart rate, physical profile and goals.

In some embodiments, the exerciser visits a central computer site through any compatible networked device, including computer, telephone, personal organizer, etc. and completes a list of specifications regarding age, gender, health and exercise goals. The central computer site then creates a profile for the exerciser along with a customized exercise program.

A compatible electronic hardware component, or “Bug” is obtained by the exerciser. Each Bug can contain a unique identifier. The exerciser associates that Bug ID with her exercise profile by actuating a user controlled input mechanism on the Bug, such as a mechanical switch, in coordination with instructions given by the central computer site. Once the association is complete, the Bug remains persistently associated with that user's profile and program.

The Bug communicates with the network through wired or wireless communication. Whenever the Bug comes within the communication range of a wireless access point, or connects to a wired interface, the Bug automatically communicates with the central computer site to download any changes in the exerciser's profile or program and upload records regarding the exerciser's physical activity.

The Bug is capable of wirelessly communicating with an external heart rate monitor or the Bug can have a heart rate monitor integrated within itself. The Bug optionally interfaces with other physical monitoring devices including, but not limited to, body temperature, respiration rate, air temperature and humidity. The Bug also communicates with compatible pedometer, compass or GPS units for measuring speed, direction and distance traveled. The Bug includes ways to calculate current heart rate. For as long as the exerciser carries the Bug and it is enabled, her physical activity, including heart rate data for each time period and any optionally monitored parameters, is monitored and logged.

When the exerciser activates the Bug's coaching mode, the Bug can implement an exercise schedule as dictated by its latest central computer site download. The Bug instructs the exerciser, through any combination of visual, aural, and tactile means, to exert more or less effort according to the current heart rate of the exerciser as compared to the heart rate specified by the central computer site created exercise program. The exerciser may deactivate the coaching mode at any time.

The exerciser also benefits by wearing the Bug outside of exercise sessions. For example, a resting heart rate, which is the rate at which the heart beats just after awakening from a night's sleep, is a good relative indicator of mental and physical stress as well as a good relatively indicator of overall fitness. Logging resting rates at regular intervals over extended periods enables better understanding of the exerciser's physical condition.

The Bug is capable of configuring and monitoring exercise performance on compatible exercise equipment including treadmills, elliptical trainers, stair machines, free weights, weight machines, stationary bicycles, rowing machines and other such devices found in the home and in athletic facilities.

When the exerciser selects a piece of such equipment to use, in some embodiments of the invention she activates a configuration function on the exercise machine, which causes the machine to establish a communication association with the Bug. Once associated, the Bug transmits signals to the exercise machine to increase or decrease the exercise workload in accordance with the dictates of the exercise program it carries and the current heart rate of the exerciser. The exerciser can, at any time, override the Bug's control of the exercise machine and modify the intensity of the workout, including terminating the exercise session. The Bug records all exercise activity on each machine, including user-defined preferences and user-commanded intensity changes. The Bug uploads the recorded data when it next communicates with the central computer site.

Alternative Bug embodiments provide an input mechanism on the Bug itself by which the user can configure heart rate and, optionally, exercise goals without need for connecting to a central computer site. This embodiment communicates through a wired or wireless connection to a unit of compatible exercise equipment. Once the connection is made, the Bug guides the exercise equipment's workload settings to keep the exerciser's heart rate within a desired range.

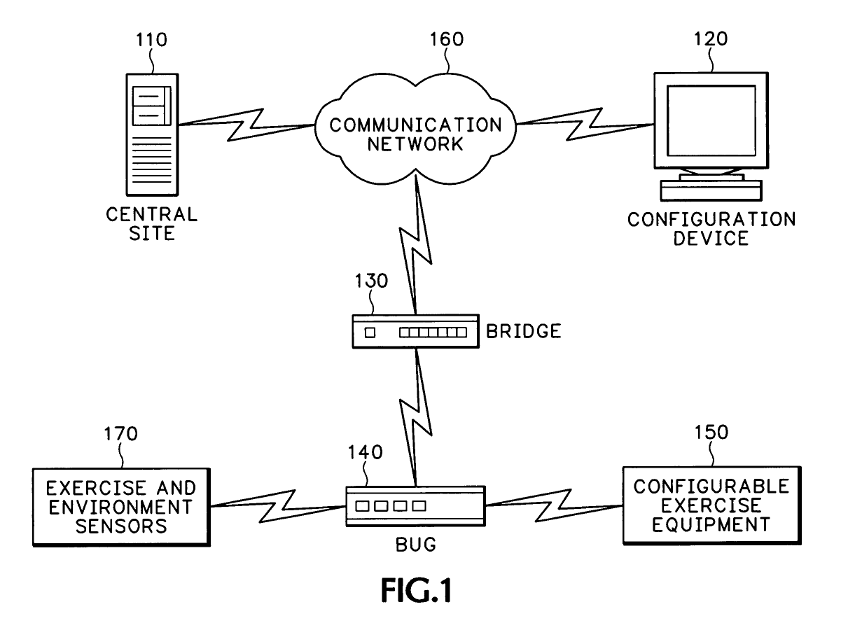

FIG. 1 illustrates an example exercise network 100 according to embodiments of the invention. In FIG. 1, the exercise network 100 includes a central site 110, a configuration device 120, a bridge 130, a Bug 140 and a piece of configurable exercise equipment 150. A communication network 160 couples the central site 100, the configuration device 120 and the bridge 130, while extensions of the communication network 160 or a separate communication network couples the bridge 130 to the Bug 140 and to the exercise equipment 150. Exercise and environment sensors 170 provide input about the exerciser, the exertion level of the exerciser, and factors about the exercise environment to the Bug 140. Of course, not all pieces illustrated in FIG. 1 are required for all embodiments of the invention.

Central Site, Network and Configuration Device

The central site 110 can include a computer system, such as a personal computer (PC), that is equipped with non-volatile storage, such as a hard disk drive, and a network interface. The communication network 160 can be any way to convey information from one device to another and those skilled in the art recognize that a wide range of wired, wireless and combined wired and wireless technologies are appropriate for the exchange of such data. In the preferred embodiment, the network interface in the central site 110 is an Ethernet interface that connects to the communication network 160, such as the Internet, through a firewall. Ethernet equipment is widely commercially available and products for both wired and wireless implementations are reliable. Other networking methods can be equally substituted for Ethernet, such as Token Ring, FDDI, hard wired serial port, dial-up modem, USB, Infrared, Firewire and other network types of wired, wireless and any combination of wired and wireless communication.

The Internet is readily available throughout much of the world and is useful in connecting networks separated by distance. Usage of all available wired and wireless network technologies, including those developed in the future is anticipated by this invention.

The central site 110 preferably maintains an account for each individual exerciser. The account includes information regarding the exerciser's profile, such as: age, fitness level, maximum heart rate, current workout regimen, exercise machine configurations and an exercise log. The central site 110 can obtain such information from the user via the configuration device 120 and from information gathered by the exerciser's Bug 140, as described below.

When an individual exerciser begins to use embodiments of this invention, she uses the configuration device 120 to establish her account with the central site 110. The purpose of the information stored in the account is to determine a maximum heart rate value for the exerciser as well as to store fitness goals and other relevant exercise information.

If the exerciser already knows her maximum heart rate, its value can be entered directly through the configuration device 120. The exerciser is also given the option to specify other parameters such as gender, age, general fitness level, number of workouts per week, workout time limits, if any, and fitness goals. If the exerciser did not specify a maximum heart rate, one is calculated for her using a formula such as MAXIMUM HEART RATE=220−AGE. In the preferred embodiment, the exerciser can use a more sophisticated formula such as MAXIMUM HEART RATE=205−(AGE/2). In the latter formula, an additional five counts are added if the exerciser is male and five more counts are added if the exerciser's fitness level is considered high. Other formulae may be used. Methods of calculating or projecting an exerciser's maximum heart rate are gradually improving and such techniques are well known to those skilled in the art.

Software on the central site 110 and/or the configuration device 120 then converts this data into a workout regimen, which is stored within the user's account on the central site. In the preferred embodiment, the central site 110 is reached through an Internet browser. The central site 110 then either calculates the workout regimen within itself or downloads a temporary program, such as a Java or JavaScript routine, which will accomplish the calculations. The resulting workout program is stored on the central site 110.

In small systems, the functions of both the central site 110 and the configuration device 120 may be implemented on a single computer.

The configuration device 120 can be a PC or public Internet terminal, for instance. Alternatively, a cell phone, Personal Data Assistant, such as a Palm Pilot or any other device with appropriate network connections and user interface may be used as the configuration device 120.

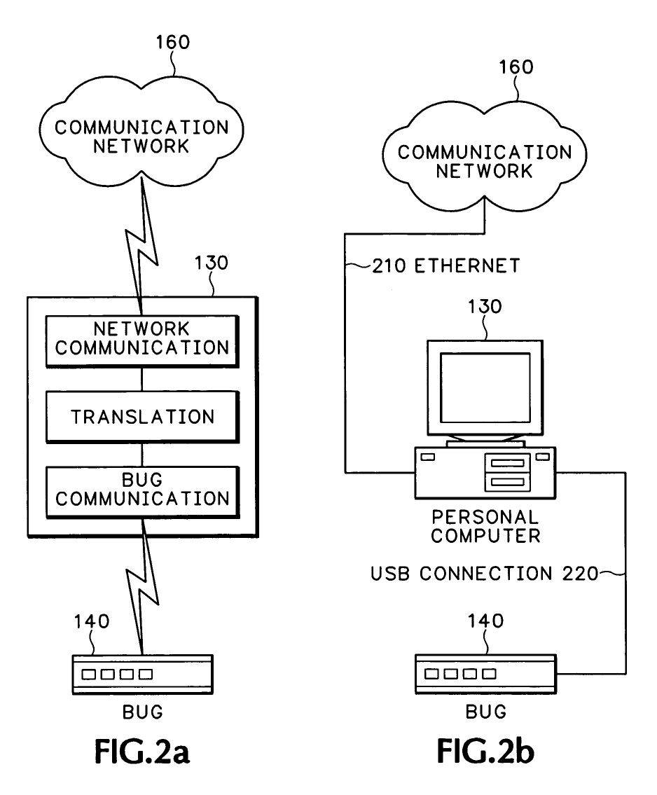

FIG. 2A illustrates a functional block diagram of an example bridge 130 according to embodiments of the invention. The bridge 130, translates a protocol used by the Bug 140 into a protocol used by the network 160. If the two protocols are the same, a bridge 130 is unnecessary. However, because the Bug 140 is portable, and is preferably wireless, battery powered and has the smallest size possible, such factors could preclude the Bug 140 from supporting a traditional wireless protocol, such as the Wi-Fi or 802.11 standards that are now in widespread use.

A preferred wireless communication protocol uses a minimum of power, and automatically senses and attaches itself to any compatible network. The preferred protocol also includes methods for avoiding interference and supports message error checking and automatic retransmission of messages that are not properly received. The well-known ZIGBEE protocol, which is based upon the IEEE 802.15.4 standard, meets many of the preferences for an appropriate protocol for implementing this invention. The ZigBee standard supports spread spectrum transmissions on several frequencies: 2.4 GHz (10 channels), 915 MHz (6 channels) and 868 MHz (1 channel). Spread spectrum technology is useful in minimizing susceptibility to RF interference from other devices and the generation of RF interference to other devices. The ZigBee standard minimizes power consumption while supporting data transmission rates from 20,000 data bits per second up to 250,000 data bits per second.

While any RF frequency could be technically used, laws governing RF spectrum use along with potential interference from other spectrum users such as radio and television broadcasts, make the use of most frequencies impossible from a legal and interference perspective. In addition, power management, communication protocols and the frequency hopping techniques necessary to implement spread spectrum communication are difficult problems that are well known in the communication industry. Standards such as ZigBee let semiconductor companies develop complete implementations on integrated circuits and offer them to the marketplace at low cost. Though the preferred means of communication utilizes RF wireless technology, infrared, ultrasonic and other wireless means are feasible and may be acceptable tradeoffs.

A wired bridge may also be used. An example of a wired bridge 130 is illustrated in FIG. 2B. In this example exercise network 100, a wired bridge interface 130 can be implemented by a PC. In FIG. 2B, the Bug 140 communicates with the bridge 130 over a wired communication interface such as a standard computer USB (Universal Serial Bus) connection. The Bug 140 is connected to a personal computer via USB port. A software application on the PC then translates data traveling to and from the Bug 140 into data to and from the central site 110 via the communication network 160. While this illustration represents the use of Ethernet (210) as the connection between the communication network 160 and the PC 130, and represents the use of USB (220) as the connection between the PC 130 and the Bug 140, other protocols are contemplated. For example, the connection between the PC 130 and the communication network 160 could be through a dial-up phone line while the connection between the PC 130 and the Bug 140 connection could be RS-232 port, FireWire or other such interface. Additionally, the exercise network 100 could use a combination of wired and wireless technologies, for instance by having a wired protocol between the communication network 160 and the bridge 130 and by having a wireless protocol between the bridge 130 and the Bug 140.

The manner by which the Bug 140 communicates is irrelevant to this invention.

Bug

The Bug 140 is a portable electronic circuit. Typically, the Bug 140 is worn on the clothing or held in a pocket of the person exercising. The Bug 140 is designed to monitor one or more parameters about the exerciser and/or the environment they are working. These parameters may be recorded.

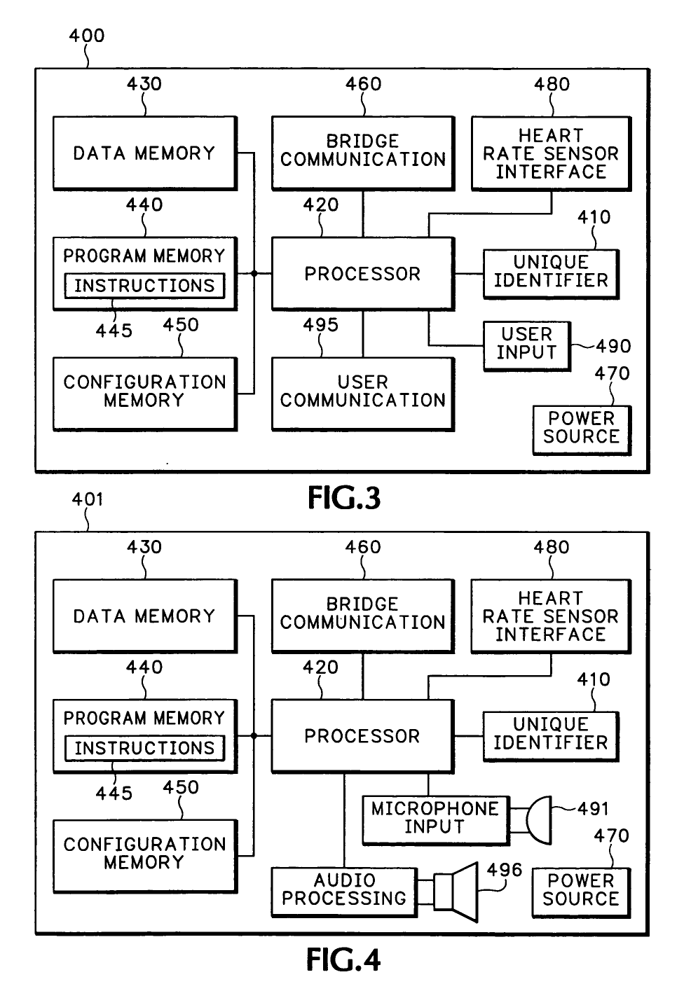

FIG. 3 illustrates a functional block diagram of an example Bug 400. The Bug 400 includes a unique identifier 410, processor 420, data memory 430, program memory 440, configuration memory 450, software instructions 445 stored within the program memory 440, bridge communication circuitry 460, a power source 470, a heart rate sensor interface 480 user input capability 490, and a mechanism 495 to communicate information to the user. Communicated information can include instructions regarding configuration and operation of the Bug 400 as well as coaching commands such as, but not limited to, “begin exercise”, “increase exercise intensity”, “decrease exercise intensity”, “maintain exercise intensity” and “stop exercise.”

In the preferred embodiment, the unique identifier 410 is accomplished by storing a unique code pattern within the configuration memory 450 at time of manufacture. An alternative implementation installs a separate silicon ID device such as Maxim Integrated Products DS2411 Silicon Serial Number, which includes an embedded 64 bit unique serial number. Those skilled in the art will recognize many such alternative approaches so long as the identifier is unique and protected from corruption or accidental erasure.

In a preferred embodiment, the processor 420 can be implemented by of a Renesas H8/3664N, which offers a 16 bit processor 32K of program memory and 6K of data memory, including 4K that is implemented as non-volatile EEPROM. The H8/3664N provides FLASH memory for its program space, allowing program code to be updated after manufacture of the Bug 400. In this implementation, one block of the FLASH memory is designated as configuration memory for storing calibration and option configuration parameters that change rarely after manufacture. This same section can also house the unique identifier 410. The H8/3664N offers small size and adequate processing power. Those skilled in the art will recognize that final processor selection is dependent upon the exact peripherals and configurations chosen. There are many suitable processor alternatives, including units that utilize masked ROM to hold the program code and unique identifiers. The art of processor design is changing rapidly as memory space and processing power increase while and physical size and power consumption diminish. Such trends are expected to continue and will allow an even richer range of processor choices than is presently available.

Wireless or wired communication circuitry 460 is implemented in a form compatible with that used by the bridge 130.

The power source 470 can be a battery having a sufficient size to provide power for several hours or days of operation. The chosen battery size is influenced by where the Bug 400 is mounted and the power requirements of supported circuitry. An improved power circuit includes a secondary battery or low-leakage capacitor, such as an NEC SuperCap, to retain data memory contents when the battery is exhausted or removed for replacement.

FIG. 4 is a functional block diagram of a Bug 401 that includes a microphone input 491 and an audio processing/speaker unit 496. Other components of the Bug 401 can be the same as is illustrated in FIG. 3. The audio processing unit 496 can produce audio tones and preferably a synthesized human voice as a way to communicate coaching instructions to the user. The audio signal is created by an earpiece, audio speaker or other audio transducer. In the preferred embodiment, five human voice commands are digitized and stored within the program memory 440 or data memory 430. The preferable digitized words are “start exercise”, “end exercise”, “increase”, “decrease”, and “maintain.” The microphone 491 is also set to receive voice or other audio commands. Such an audio input is not limited to this embodiment but may be used with any other form of user communication. Similarly, any type of user input can be used with the audio output communication depicted here.

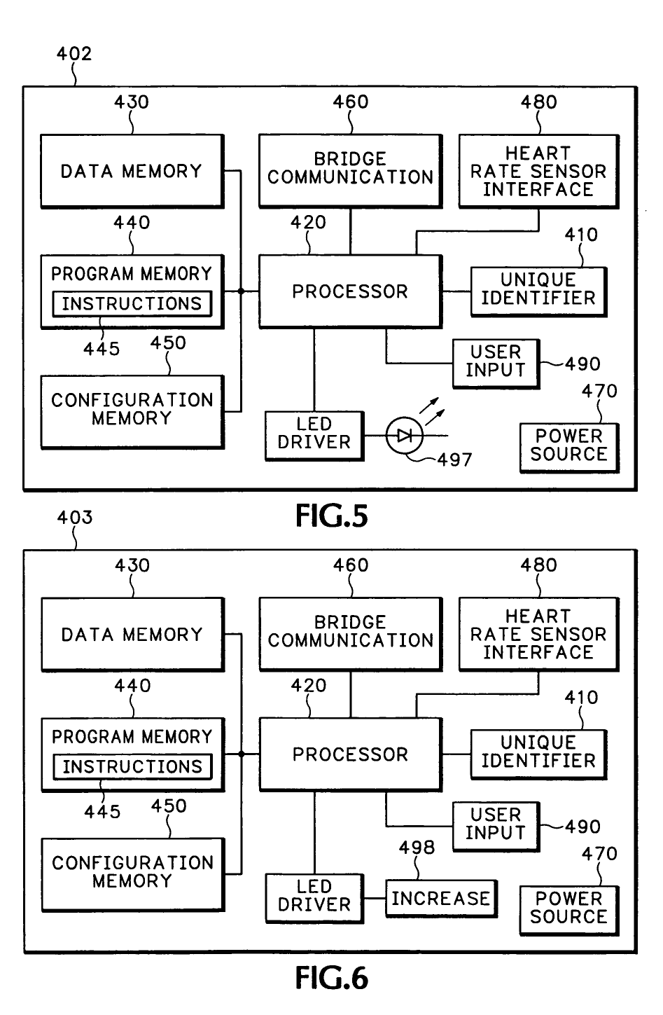

FIG. 5 is a functional block diagram of a Bug 402 illustrating an LED driver and LED 497 as a form of user communication. Other components of the Bug 402 can be similar to those illustrated in FIG. 3. The LED 497 provides a visual form of communicating coaching instructions. In the preferred embodiment, the LED 497 includes two colors, e.g., red and green, though other numbers of colors, color combinations, flash intervals and durations are equally possible. The “begin exercise” command is communicated by periodically flashing the green LED 497 at a steady rate; in this embodiment, one 20 ms flash every second. In this embodiment, the flashing green light that indicates the exercise has begun also indicates that the exercise intensity should be increased. The “maintain intensity” instruction can be represented by alternately flashing the red and green LEDs 497 in 20 ms pulses at one-second intervals. “Reduce intensity” is indicated by 20 ms pulses of red light from the LED 497 every ½ second. “Stop exercise” is indicated by 50 ms flashes of red light at ¼ second intervals.

FIG. 6 is a functional block diagram of a Bug 403 that includes a Liquid Crystal Display (LCD) driver and LCD screen 498 as a form of user communication. Other components of the Bug 403 may be similar to those illustrated in FIG. 3. The LCD screen 498 communicates coaching instruction. In the preferred embodiment, the words “Start”, “End”, “Increase”, “Decrease” and “Maintain” are spelled out on the display face 498 to communicate the particular command. These commands can easily be represented by other words, including words chosen from other languages. It is also possible to use visual symbols or number codes to represent the coaching instructions.

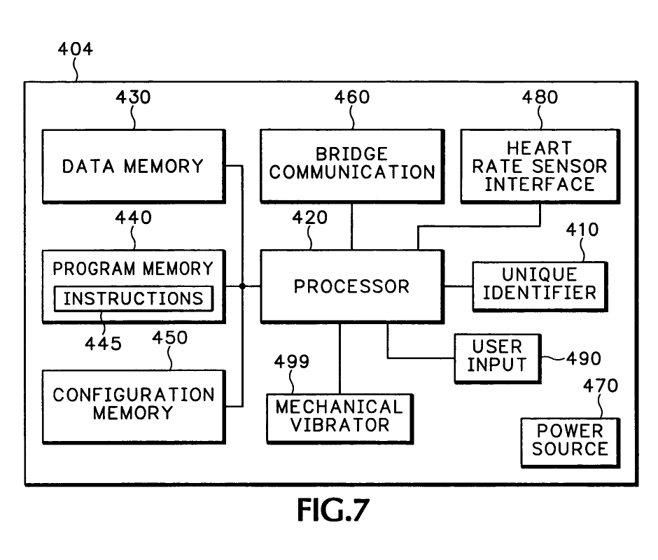

FIG. 7 is a functional block diagram of a Bug 404 that includes a tactile form of user communication, such as a mechanical vibrator 499. Other components of the Bug 404 may be similar to those illustrated in FIG. 3. The vibrating mechanism 499, similar to that used to create a silent, vibrating mobile phone ringer, is placed in proximity to an area of the exerciser's body. By energizing the mechanical vibrator at particular times, messages can be communicated to the exerciser. When the exercise is to begin, for example, two pulses of vibratory motion are emitted from the vibrating mechanism 499, each lasting for ½ second and spaced ½ second apart. When exercise intensity is to increase, a pairing of pulses may be emitted every ten seconds. When exercise intensity is within the desired range, no vibrations may be are emitted. When exercise intensity is to decrease, a series of three one-half second vibratory pulses may be emitted within ½ second of each other. This pattern repeats every five seconds until the heart rate of the exerciser falls beneath the threshold level. The vibratory pulse lengths and timing patterns represented here are the currently preferred embodiment though other vibratory patterns of different timing, amplitude and duration may be substituted.

FIGS. 4 through 7 are the currently preferred embodiments by which coaching instruction can be communicated to the exerciser. Other audio, visual and tactile interfaces are possible and contemplated by this invention.

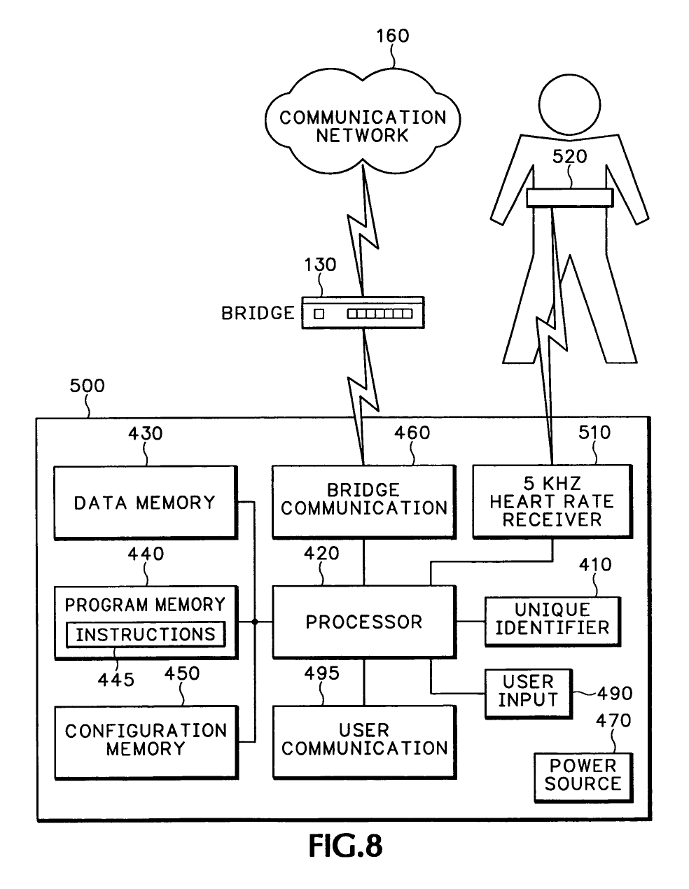

The Bug 140 can be configured with a self-contained or external heart rate sensor. A preferred embodiment, FIG. 8, is a functional block diagram showing an embodiment of the Bug 500 that includes a 5 KHz receiver 510 as the Heart Rate Sensor interface 480 of FIG. 3. The receiver 510 is inductively coupled to receive pulses generated by readily available heart rate sensor chest strap 520, such as the Polar Electro Corporation's T31 model.

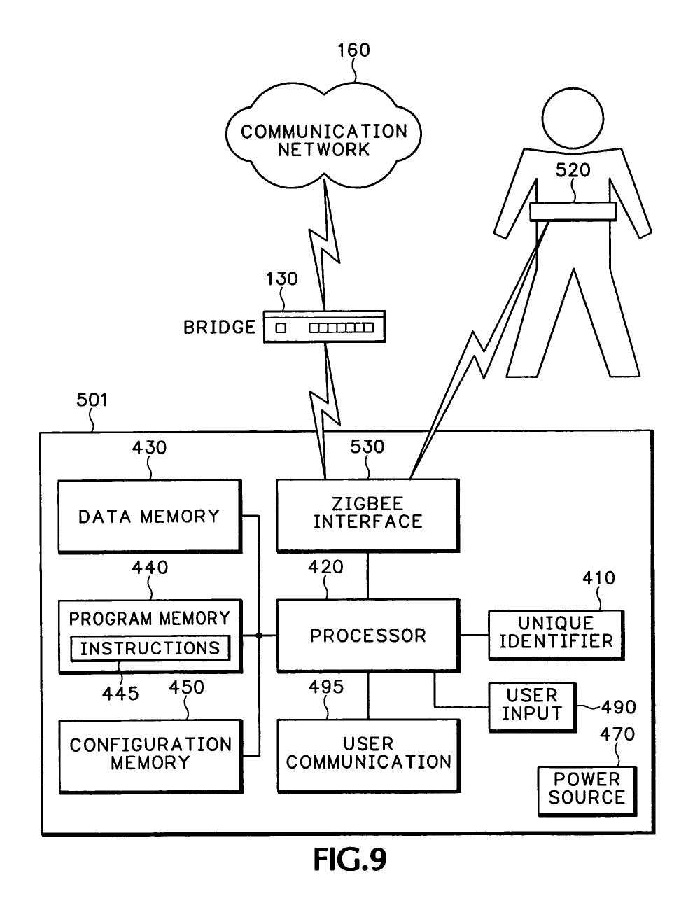

In an embodiment illustrated in FIG. 9, a Bug 501 includes a ZigBee interface 530, which functions as both a receiver from the heart rate sensor chest strap 520 as well as a communication link to the bridge 130. The ZigBee interface 530 receives heart rate sensor emitting signals detectable by the same wireless communication circuitry that the Bug 501 uses for network communication, in this case the ZigBee RF wireless protocol. Such a heart rate sensor 530 could monitor the heart's electrical signals, whether mounted on a chest strap 520 or located elsewhere in the exercise network 100. An alternative technology is to detect heart rate by sensing the flow of blood through capillary or artery within the body. Such detection means are generally implemented through optical technology in which infrared, visible light or other radiation is emitted into the body and reflected or blocked by blood pulsating through a blood vessel. A photo sensor detects the light that is reflected or passes through the skin, depending on configuration of sensors, in such a manner that the light signal pulses on and off in response to blood flowing in said blood vessels. Heart rate sensing technology based upon light reflected or blocked by blood flow is well known to those skilled in the field.

Other heart rate sensors are constructed of pressure sensors that detect the physical pressure of blood flowing through the body. Such technology is also well known to those skilled in the art. Embodiments of the invention can utilize any method of heart rate detection whether sensed by electrical, optical, mechanical pressure or other means.

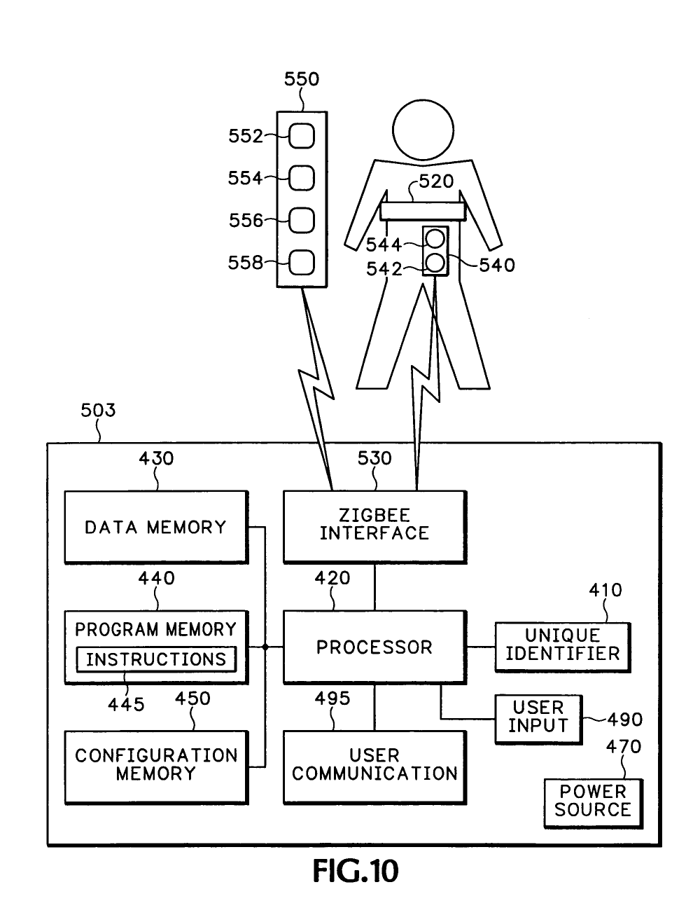

FIG. 10 illustrates a Bug 503 that includes an additional subsystem 540 for sensing data about the exerciser. In the embodiment shown in FIG. 10, the subsystem 540 illustrates an exerciser body temperature sensor 542 and an exerciser respiration rate sensor 544. Additionally, the Bug 503 includes a set of sensors 550 for detecting environmental factors, such as a surrounding air temperature sensor 552, humidity 554, direction of travel 556, and speed of travel 558. To minimize Bug 503 size, the currently preferred embodiment communicates with the subsystems 540, 550 through wireless communication, preferably using the same wireless protocol by which the Bug 503 communicates with the bridge 130 illustrated previously. As more powerful and smaller electronic circuitry is developed, the subsystems 540, 550 can eventually be integrated within the Bug 503 without increasing its size beyond a range that the exerciser finds comfortable. Such integration is an anticipated part of this invention.

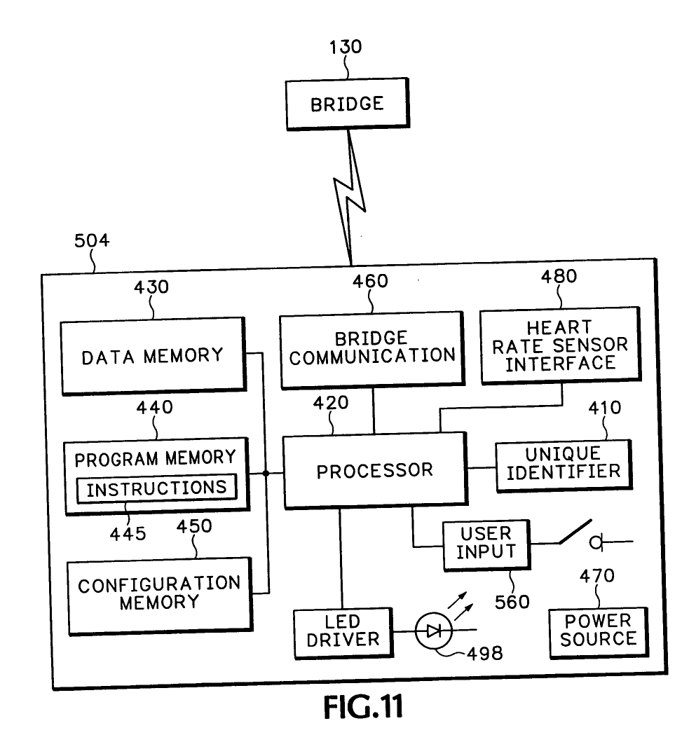

In another embodiment, a Bug 504 includes one or more user-actuated inputs 560 as illustrated in FIG. 11. The inputs 560 may be a mechanical switch or switches, the input of which used by the Bug 504 to affect user instructions for functions such as enable/disable, audio volume, processor reset or to cause an association with the Bug's unique identifier 410. FIG. 11 represents the Bug 504 with one mechanical switch 560 and a single visual indicator, implemented by the LED driver and LED 498, similar to the LED driver and LED 487 described above with reference to FIG. 5. The LED 498 is used to indicate device status.

The Bug 504 can be programmed to allow multiple functions from the single input 560. In this embodiment, activating the input 560 continuously for three seconds toggles the Bug 504 between its enabled and disabled states. The disabled state preserves battery life when the Bug 504 will not be used for an extended period. In another function, activating the input 560 with five sequential activations, each of less than ½ second duration and each within ½ second of the other, causes an “association mode” to begin, which allows the central site 110 (FIG. 1) to associate the Bug's 504 unique ID 410 with the exerciser's record. In still other functions, if the input 560 is activated for a single ½-second interval, it will toggle the coaching mode on and off. Other input combinations are possible and contemplated by this invention. Such multi-function usage of an input device 560 is well known to those skilled in electronic or embedded software design.

The device status LED 498 can be embodied in a single physical device containing two color indicators, blue and yellow, though other colors are equally useful. When the Bug 504 is enabled, the blue light flashes for approximately 20 ms every three seconds. Such a low duty cycle flashing scheme is employed to minimize power consumption. When the Bug 504 comes within range of the bridge 130, the blue light and yellow light alternately illuminate at three second intervals for 20 ms each, informing the user that a network connection has been established without increasing overall power consumption.

In some implementations of the Bug, it is desirable to utilize the coaching instruction communication facility, several embodiments of which were discussed above, to represent the device status. Those skilled in the art understand that each indicated message, no matter how communicated, must be readily differentiable from the other indicated messages.

First System Operation

The first system is a minimal group of the above-described components that can accomplish the exercise network 100 function of this invention. Such components can include (As illustrated in FIG. 1) the central site 110, bridge 130, communication network 160, configuration device 120, Bug 140 and sensors 170, which may include a heart rate monitor 610. Functions of each individual component have been previously described.

The Bug 140 can be configured to monitor, using inputs from the sensors 170, parameters such as exerciser body temperature and respiration rate, air temperature and humidity, speed, distance and direction traveled. For sake of clarity, only the heart rate function of the sensor 170 is discussed in this section, and is referenced as 610. Those skilled in the field will easily understand that methods similar to those described here for heart rate processing can be readily applied to one or more of the aforementioned parameters in addition to, or in substitution for, heart rate.

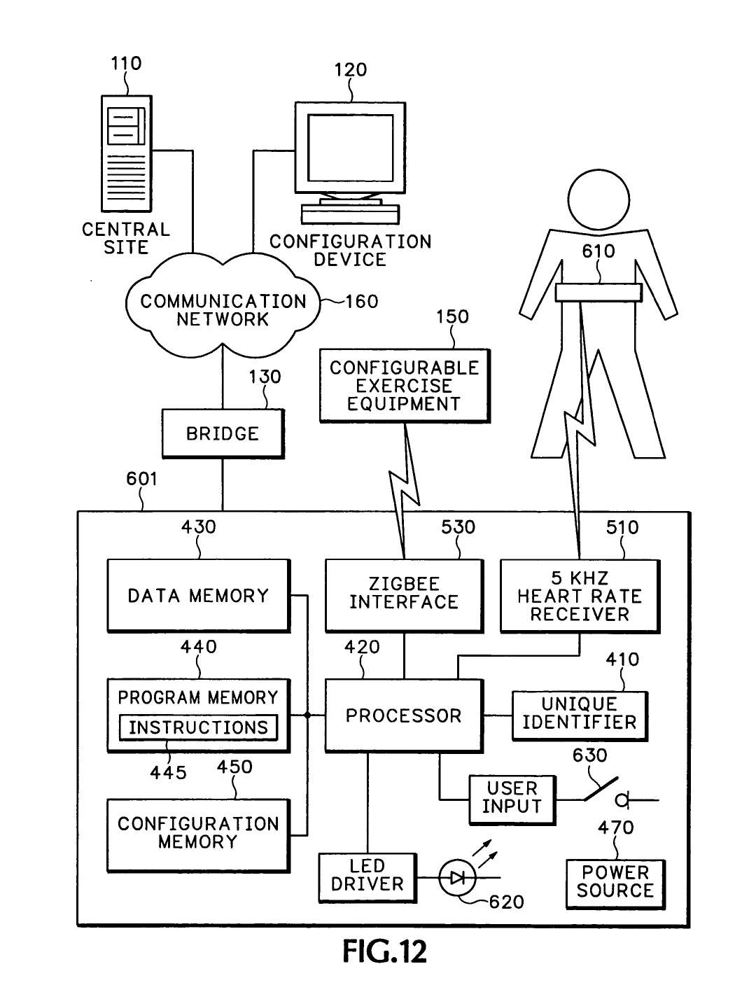

FIG. 12 represents an example Bug 601 used in this first system. This Bug 601 includes a single, three-color LED 620 to indicate both device status and coaching instruction. The single LED element 620 contains three emitting die, one each for red, green and blue. Multiple die may be activated to create color combinations, for example, if the red and green die are simultaneously energized, a yellow color results. Such multi-color LEDs are available from a variety of sources, such as Nichia Corporation's NECM325C. A single user input, in this case a mechanical switch 630, is also included. A heart rate sensor 610 is a standard chest strap and emits a 5 KHz signal pulse with each heartbeat. The Bug 601 receives these signals through inductive coupling counting each pulse as one heartbeat. The implementation represented in FIG. 12 and discussed herein is but one example of Bug implementation. Other structures, including those earlier described, can also be used without deviating from the inventive spirit herein.

Using the embodiment of FIG. 12 as an example, the interoperability of the various components of this invention can be illustrated. It is presumed that the exerciser has accessed the central site 110 through the configuration device 120 and created an account, as earlier described.

During the process of creating or modifying her account, the exerciser must associate the Bug 601 with her account identifier so that information can automatically transfer between the two.

The Bug 601, as earlier described, contains a unique ID 410. The ID length and composition is not material to this invention so long as the ID is unique among all Bugs 601. Each account is assigned an identifier, which may be of any length or composition.

The Bug's unique ID 410 is associated with an account ID on the central site 110. In the preferred embodiment, this is accomplished by storing the Bug's unique ID within the exerciser's record on the central site 110 and storing a copy of the exerciser's central site 110 account identifier in the Bug's configuration memory 450. Only one of these two associations is necessary to establish a unique relationship, but by doing both, a measure of redundancy is provided. Other association techniques may be used, the exact manner of which is unimportant.

The association mechanism begins when the exerciser first creates her account. The central site 110 initially marks the account as “unassociated”. The central site 110 then scans its network for any connected but unassociated bugs. If none is found, a message is sent to the configuration device 120 telling the exerciser to connect her Bug 601 to the network. Presuming a wireless network is used, using the ZigBee protocol for example as described above, the connection is accomplished simply by placing the Bug 601 within range of the bridge 130. With ZigBee, this range is typically anywhere within 20-30 feet of the bridge 130. If a wired network such as USB is used, the connection is accomplished by plugging the Bug 601 into a USB connector on the bridge 130 or the PC used to affect the bridge function.

When the Bug 601 is connected to the communication network 160, the Bug searches out the central site 110, such as by searching for an IP (Internet Protocol) address of the central site 110 that has been pre-programmed into the Bug 601 or, more preferably, by searching for the central site's domain name, which can also preprogrammed into the Bug's memory. Searching for the domain name provides the specific IP address of the central site 110 through one or more Domain Name Servers. Both processes are well known to those skilled in the field.

When the Bug 601 establishes a connection to the central site 110, its LED 620 begins flashing alternately between red and green at two-second intervals, showing that a connection is accomplished. The Bug 601 and central site 110 then communicate and the Bug 601 identifies itself by its unique ID 410. The central site 100 compares the Bug's ID 410 to a list of IDs associated with various accounts. When no associated account is found, it adds the Bug's ID 410 to its list of unassociated Bugs.

Presuming one or more unassociated Bugs is on the network, the central site now attempts to associate each Bug 601 with an account. It is possible that an unassociated Bug 601 is on the network but the exerciser to whom it belongs is not currently attempting to create or modify her account. It is further possible that multiple unassigned Bugs 601 are on the network along with multiple exercisers attempting to create or modify their account.

To overcome the chance of improper association, the central site 110 sends a message to one exerciser that asks her to put her Bug 601 into the association mode. In this embodiment, the Bug 601 is placed into the Association mode by, for example, pressing the input switch 630 five times with less than ½ second on each press and no more than ½ second between each press. The actual method for entering the association can be made in any acceptable way, and multiple ways to enter the association mode are possible.

Once the Bug 601 has entered the association mode, it sends a message to the central site 100 that the Bug 601 is ready to be assigned. The Bug 601 now flashes its LED 620 at one-second intervals alternating between red, green and blue colors to verify to the exerciser that an association is in progress.

It is possible that more than one bug 601 can simultaneously be in association mode with multiple exercisers attempting to complete the association of Bug with account record. To insure proper association, the central site 110 sends a message through, for example, the configuration device 120, to an exerciser asking her to press the input switch 630 one time within the next ten seconds. When the exerciser presses the switch 630, a message is sent from the Bug 601 to the central site 110 confirming the button press. The central site 110 then makes a preliminary association between the account of the exerciser that was instructed to press the input switch and the unique ID 410 of the reporting Bug 601. The central site 110 then sends a preliminary confirmation message to the Bug 601, causing it to flash the blue LED 620 on and off at ¼ second intervals.

The central site 110 sends a query message to the exerciser, through the configuration device 120, asking her to verify that her Bug 601 is flashing blue at a rapid rate. If the exerciser responds affirmatively, the association is completed. If the confirmation does not come through within a specified time—for example within 10 seconds—the process is repeated.

The above described manner of association is but one of many possible techniques. This invention can use any association technique so long as it eliminates all ambiguity in the association process.

Once an association is made, the Bug 601 initiates communication with the central site 110 each time the Bug 601 is connected to the communication network 160. New exercise regimen and compatible exercise equipment configurations can be copied to the Bug 601 and stored in one of its memories, 430, 440, 445, or 450. Any new exercise logs or compatible exercise equipment logs may also be transferred back to the central site 110. Once the Bug 601 completes and confirms the transfer of its log and configuration data to the central site 110, the data memory in which it was stored is made available for new log and configuration data.

When the Bug 601 is placed into the coaching mode, it determines the exerciser's current heart rate, as determined by counting pulses received from the Heart Rate Sensor 610. These pulses are filtered for noise, averaged and converted to a number representing beats per minute of the heart.

The current beats per minute measure is compared to the target rate of the exercise profile copied from the central system 110. Based upon how the current heart rate compares to the target rate, the exerciser is instructed to increase, maintain or decrease exercise intensity to keep the current heart rate equal to the target rate, as described above. In practice, a range of beats is created around the target rate to make coaching instruction more consistent. For example, if the target rate were 140 beats per minute, the exerciser would be told to maintain the current exercise intensity for so long as the current heart rate was between approximately 135 and 145 beats per minute. When the exercise period has expired, the exerciser is told to stop the exercise.

During the exercise period, heart rate activity is logged into one of the Bug's 601 memories, such as the data memory 430, at regular intervals for later reporting to the central site 110.

Configurable Exercise Equipment

Embodiments of the present invention are also useful in configuring and controlling exercise equipment that is compatible with the Bug's protocol. Such exercise equipment can include, but is not limited to, treadmills, stair machines, elliptical trainers, stationary bicycles, climbing machines, weight machines, rowing machines and free weight equipment. Though the following discussion describes a treadmill as the compatible exercise equipment, the invention contemplates usage with all types of exercise equipment as previously described. Those skilled in the art will readily understand how to adapt the present invention to exercise machines other than treadmills.

At the most elementary level, the Bug 601 takes control of compatible exercise machine parameters that determine exertion level. When the Bug 601 is used with a compatible treadmill 150, the treadmill speed is controlled by the Bug's own processor 420. If the Bug 601 senses, based upon the exerciser's heart rate, that exertion level should increase because the exerciser's heart rate is below the target, the Bug 601 sends a command to the treadmill 150 telling it to increase speed by a specific amount. After a time, for example 30 seconds, the Bug 601 again tests the exerciser's heart rate against the target. If the heart rate is still below target, the treadmill 150 is again instructed to increase speed by another amount.

If the heart rate is within the target zone, the treadmill 150 is instructed to maintain speed. If the heart rate is above target, the treadmill 150 is instructed to slow by a given amount. When the exercise period ends, the treadmill 150 is instructed to slow and stop.

Such control frees the exerciser from worry about adjusting controls on the treadmill 150 and removes any need for the exerciser to monitor her heart rate, though that information is optionally displayed, either through the treadmill 150 or through one of the ways of user communication 495 of the Bug, if the exerciser wishes to monitor it.

This system is especially useful in warm-up periods wherein heart rate ramps up to the target rate over a time. A typical warm-up period is fifteen minutes during which the exerciser's heart rate should gradually rise from its normal rate to the target rate. As an example, the exerciser has a heart rate of 75 Beats Per Minute when the workout session begins and a target of 150 BPM for the actual exercise period. The warm-up session should increase the heart rate approximately linearly from 75 BPM to 150 BPM over a period of approximately fifteen minutes, which is an increase of about 5 BPM/minute. At the end of minute one, the heart rate should reach 80 BPM and 90 BPM at the end of minute three. By maintaining such an increase, the exerciser will reach the targeted 150 BPM at the end of the desired fifteen-minute warm-up period. Of course, if other warm-up durations are desired, parameters within the bug 601 and/or central site 110 can be modified to accommodate.

Since the ramp up in heart rate is handled automatically by the Bug 601 and treadmill 150, the exerciser is free to direct her attention elsewhere. Similarly, the Bug 601 and exercise machine 150 work in tandem to implement a cool-down period in which exertion levels are slowly reduced until the exerciser reaches a desired minimum rate at the end of the designated cool-down time.

The Bug 601 can optionally monitor how an exerciser's heart rate responds to a unit of change in the exercise machine's 150 workload and use that response rate to better match increases and decreases in workload to the hart rate changes required to reach the goal range. For example, assume Mary has a heart rate of 105 after sixty seconds on the treadmill 150 at 4.7 miles per hour. When the treadmill 150 speed is increased to 5.0 miles per hour, her heart rate rises to 120 after sixty-seconds of acclimatization. Doug has a heart rate of 100 at 4.7 miles per hour and his rate increases to 105 after sixty seconds at 5.0 MPH.

If, during a coaching session, Mary's heart rate were five beats per minute lower than the goal, the Bug 601 would instruct the treadmill 150 to increase its speed by 0.1 MPH and then wait for Mary's heart rate to stabilize. If Doug's heart rate were 5 beats per minute below the goal, the Bug 601 would instruct the treadmill 150 to increase speed by 0.3 MPH and then wait for Doug's heart rate to stabilize. The Bug 601 can profile its exerciser's heart rate changes across all speeds and conditions. Such information is then used for efficient control of the exercise equipment's workload to achieve the desired heart rate goal with a minimum of overshoot and undershoot. The Bug 601 can update its user profile dynamically as each exerciser's response to workload changes will vary in proportion to their fitness level, fatigue level and general health.

Many treadmills 150 offer incline controls as well as speed. Higher inclines offer greater exercise intensity. The exerciser can choose to use the incline control just by pressing the incline buttons on the treadmill 150. If the exerciser's heart rate increases past the target zone, the Bug 601 can reduce treadmill speed to compensate. Alternatively, the Bug 601 can control both speed and incline to match the fitness goals of the exerciser. The Bug 601 can be configured to control any number of machine parameters.

The exerciser can manually override the Bug's 601 commands that it sends to the treadmill 150. If the exerciser feels tired or develops a muscle ache, she can use the treadmill controls 150 to slow the speed to her desired level. She may also increase treadmill speed beyond the target rate as well.

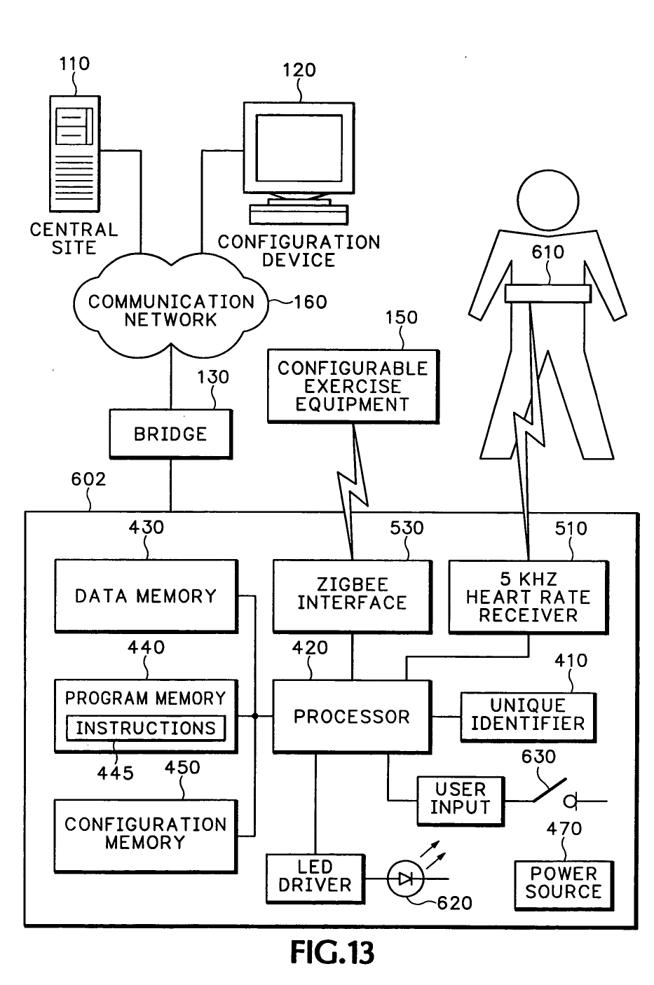

In the preferred embodiment, the treadmill 150 communicates with the Bug 602 utilizing the same interface as the Bug 602 uses to communicate with its bridge 130 though other interface techniques are possible. FIG. 13 illustrates.

In a busy health club, there can be many exercisers, many Bugs 602 and many controllable machines 150. It is important that the proper Bug 602 controls the correct exercise machine 150. The ZigBee protocol includes techniques for automatically communicating with a number of devices simultaneously, removing the concern for interference from multiple devices talking at once.

In the preferred embodiment, the Bug 602 automatically establishes a communication session with each compatible exercise machine 150 within its range. An exerciser's Bug 602 could easily become associated with not only the treadmill 150 she is standing upon but also the treadmill to her immediate left and right as well.

When the exerciser presses the start button on her treadmill 150, it sends a message to the Bug 602 saying it is the chosen machine. To confirm the association, the exerciser presses the input switch 630 on the bug. If both of these operations happen within a designated time, three seconds for example, the association is complete and the treadmill 150 begins operation while taking control instructions from the Bug 602.

If the association fails for any reason, the exerciser is informed, by the Bug 602, the treadmill 150, or both. She may then repeat the association process or instruct the exercise machine 150 to operate manually.

If a treadmill 150 start button is pressed without unassociated Bugs 602 in its range, the treadmill operates in its normal, manually controlled, mode.

Once a Bug 602 is associated with a treadmill 150, it automatically breaks its associations with all other treadmills, leaving them free for use by other exercisers. If multiple exercisers attempt to associate their Bugs with exercise equipment 150 simultaneously, one exerciser at a time is prompted to complete her association. When she is finished, the next exerciser completes her association and the process repeats until all associations are completed.

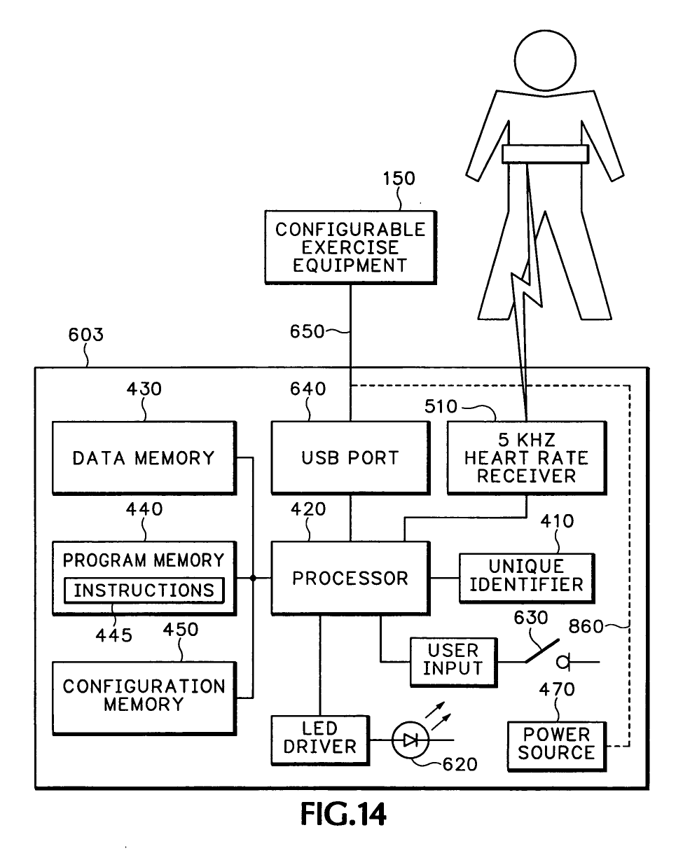

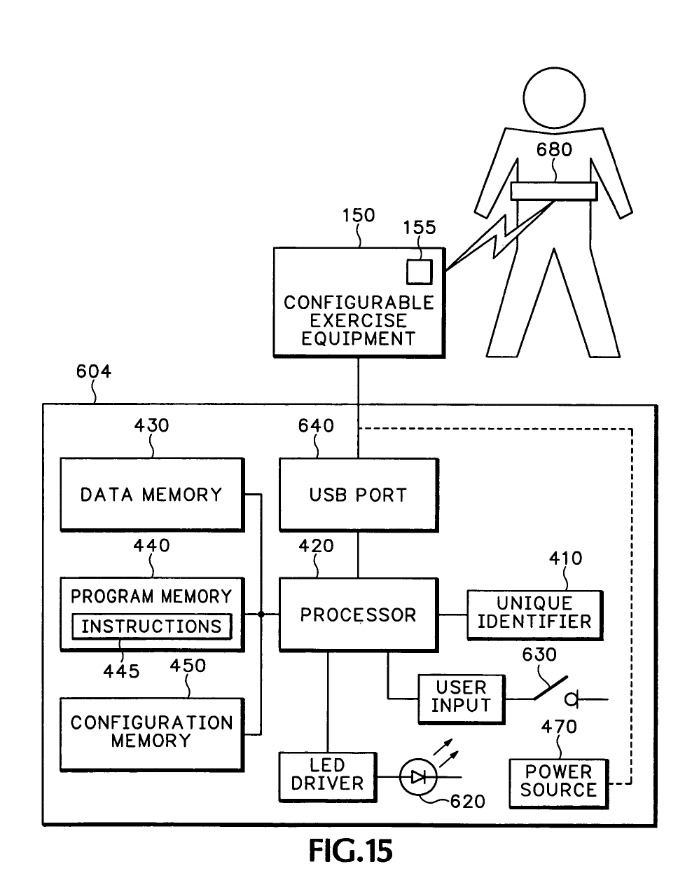

As illustrated in FIG. 14, the interface between a Bug 603 and the controllable exercise equipment 150 is a wired implementation. The Bug 603 of FIG. 14 includes a USB port 640 and a USB cable 650 coupled between the Bug 603 and the exercise equipment 150. In some embodiments, the USB port 640 can supplement or replace the Bug's 603 on-board power 470. When a physical connection such as the USB interface between the Bug 603 and the exercise equipment 150 is used, the association task is greatly simplified. With a wired interface, the machine 150 and Bug 603 begin communication immediately since there is no ambiguity of which Bug belongs to which machine.

Any method of communication that allows the treadmill and Bug to associate and communicate is satisfactory to this invention.

Another embodiment of this invention utilizes a simplified Bug 604 as shown in FIG. 15, wherein the heart rate sensor interface is not used in the Bug 604. Exerciser information can be programmed into the Bug 604 by using a PC or in other manners as described earlier. In this embodiment, a heart rate sensor 155 is built into the controllable exercise equipment 150, which, in turn, is coupled to the Bug 604. When the Bug 604 is plugged into the treadmill 150, heart rate information is sent from the treadmill 150 to the Bug 604 where workload change commands are calculated and transmitted back to the exercise equipment 150. All exercise activity may still controlled and logged by the Bug's processor 420.

In all of the above-described embodiments, functions from various components may communicate with other components in any appropriate manner. Those skilled in the art will recognize there are many ways to communicate information with controllable exercise equipment other compatible exercise equipment while retaining the advantages of a portable exercise Bug that houses information about the individual exerciser's goals and travels with her from machine to machine and activity to activity. Functions illustrated within one particular component may be performed by other components in the system. Component boundaries are for illustrative purposes and ease of discussion only, and do not necessarily mean that a particular function must be performed within those boundaries.

The invention herein described with reference to its particular preferred embodiments is not limited to such embodiments, but rather is defined by the following claims and their equivalents.

1. An exercise network, comprising:
one or more sensors structured to transmit exercise related data signals;
a portable receiver structured to accept the transmitted exercise related data signals, the portable receiver including a unique identifier; and
a central repository storing one or more information accounts, at least one of the information accounts associated with the unique identifier.

2. The network of claim 1 wherein the portable receiver is coupled to the central repository through a communication network.

3. The network of claim 1 wherein the portable receiver stores an indicator of the received data signals.

4. The network of claim 1 wherein the portable receiver comprises a communication element structured to provide feedback information to a user.

5. The network of claim 4 wherein the communication element is a controllable light.

6. The network of claim 4 wherein the portable receiver further comprises codes that, when executed, cause the communication element to provide feedback information to the user about an instantaneous exertion level of the user.

7. The network of claim 1 wherein the exercise related data signals are related to a heart beat rate of an exerciser.

8. A communication network, comprising:
a transmitter structured to transmit a signal related to an exercise parameter about a user;
one or more uniquely identified electronic units structured to receive the transmitted signal and perform data operations on the received signal;
a central server structured to store a plurality of user accounts, each account associated with one of the uniquely identified electronic units; and
a network bridge coupled between the central server and the electronic unit.

9. The network of claim 8 wherein the transmitter transmits the signal related to the exercise parameter using a first protocol.

10. The network of claim 9 wherein the electronic unit transmits a signal to the central server using the first protocol.

11. The network of claim 9 wherein the electronic unit transmits a signal to the central server using a second protocol.

12. The network of claim 8 wherein the exercise parameter is a heart rate of the user.

13. The network of claim 12, further comprising a second transmitter structured to transmit environmental factors to the electronic unit.

14. An exercise monitor, comprising:
a receiver structured to accept an exercise parameter signal about a user;
a data memory structured to store a historical record of the received signals;
a program memory structured to store codes that, when executed, cause the exercise monitor to interact with the user;
a processor structured to execute the store codes and to generate interaction signals;
a feedback mechanism structured to provide communication to the user based on the interaction signals;
a user input; and
a unique identifier.

15. The exercise monitor of claim 14 further comprising a data transmitter structured to transmit the unique identifier on a network.

16. The exercise monitor of claim 14, wherein the codes stored in the program memory cause the exercise monitor to communicate to the user the results of a comparison between instantaneous received exercise parameters and a stored parameter.

17. The exercise monitor of claim 16 wherein the exercise monitor functions to guide the user in a predefined workout.

18. A method for regulating exercise, comprising:
receiving a heart rate signal about a user;
using a unique identifier to access a network account indexed by the unique identifier;
retrieving exercise data about the user from the account, the exercise data including a target heart rate;
storing a representation of the exercise data in an electronic device proximate to the user;
comparing an instantaneous heart rate of the user to a stored target heart rate; and
providing a signal to the user indicating results of a comparison of the instantaneous heart rate to the stored target heart rate.

19. The method of claim 18, further comprising:
receiving environment parameters about the user's environment.

20. The method of claim 19 wherein receiving environment parameters comprises receiving information about one or more parameters selected from the group consisting of air temperature, humidity, direction of travel, and speed of travel.

21. The method of claim 18 wherein providing the signal to the user indicating results of the comparison comprises generating a light signal.

22. The method of claim 18 wherein providing the signal to the user indicating results of the comparison comprises generating a light signal from a multi-colored LED system.

23. The method of claim 18 wherein providing the signal to the user indicating results of the comparison comprises generating a symbol on a display.

24. The method of claim 18 wherein providing the signal to the user indicating results of the comparison comprises generating a symbol on an LCD display.

25. The method of claim 18 wherein providing the signal to the user indicating results of the comparison comprises generating an audio signal.

26. The method of claim 25 wherein generating an audio signal comprises generating a single tone.

27. The method of claim 25 wherein generating an audio signal comprises generating a synthesized voice.

28. The method of claim 18, further comprising sending a signal to a controllable exercise device based on the comparison of the instantaneous heart rate to the stored target heart rate.

29. The method of claim 28 wherein sending the signal comprises sending a signal for the exercise device to increase speed.

30. The method of claim 28 wherein sending the signal comprises sending a signal for the exercise device to increase resistance.

31. The method of claim 28 wherein sending the signal comprises sending a signal for the exercise device to increase an incline.
